(19) United States
(12) Patent Application Publication
(10) Pub. No.: US 2005/0038368 A1
(43) Pub. Date: Feb. 17, 2005

(54) Intermediate layer for enhancing comfort of a user wearing usual objects on the body

(76) Inventors: Andreas Richter, Dresden (DE); Karl-Friedrich Arndt, Merseburg (DE); Thomas Schmidt, Dresden (DE); Michael Kornert, Dresden (DE); Daniel Weber, Dresden (DE)

Correspondence Address: WILLIAM COLLARD;COLLARD & ROE, P.C., 1077 NORTHERN BOULEVARD, ROSLYN, NY, 11576, US

(21) Appl. No.: 10/492,330

(22) PCT Filed: Oct. 9, 2002
(86) PCT No.: PCT/DE02/03801

(30) Foreign Application Priority Data
Oct. 10, 2001 (DE) 101 49 952.3

(51) Int. Cl. A61F 13/00

(52) U.S. Cl. 602/41

(57) ABSTRACT

The invention concerns an intermediate layer (2) for enhancing comfort of a user wearing usual objects on or in the body. The intermediate layer comprises at least an element consisting of a heat-sensitive gel having a lower critical solution temperature (LCST). Under the influence of the user's body or skin temperature (T), the intermediate layer actively produces a constant modification of the contact point(s) between the usual object and the user's body (3).

### TECHNICAL FIELD

The invention relates to an intermediate layer for enhancing the comfort of a user wearing utility objects on or in the body.

Particularly in the case of utility objects that are worn on or in the body of a human being or an animal over an extended period of time, points that are subject to pressure stress occur at the contact points with the body, which are produced by the attachment devices, the inherent weight of the utility object, the interactions between the body and the utility object, or the like. As a result of the individual, uneven body shape, there is a non-uniform pressure distribution at the contact points, which results in reduced wearing comfort and impairment of the well-being of the user, e.g. in the form of local redness, pressure points and rubbing points, blisters, inflammations, as well as pain.

A typical reaction of a user is to temporarily relieve the stress on the pressure point, for example by changing the position of a strap or belt, moving or removing a pair of eyeglasses, changing his/her posture, or the like. However, when using utility objects that are fixed in place, such as bandages, for example, this is not possible.

### STATE OF THE ART

This problem was previously countered, in technical terms, with the use of cushioning media, such as foam materials, rubber, fluids, or air, in order to allow soft contact.

When using foam material, rubber, or the like, the contact surface is increased over the uneven body contours, and an improved adaptation to the contours and reduced stress at specific points are achieved. In addition, the rubber acts as a seal, which is necessary in the case of certain applications.

Thus, a possibility of improving bandages by means of adapting their shape to a body part is described in EP 0 852 935 A1. For this purpose, the bandage material is made to fit the special anatomical shape by means of positive or negative molding, by means of thermal deformation. In this way, disruptive connecting points can be avoided. Materials having a thermoplastic nature, such as nonwoven fabrics, woven/loom-knitted/knitted fabrics, as well as film-like plastics having a low rigidity, such as LDPE/foam material, are used as the material.

The individual anatomical imprint, however, has the disadvantage of being used only once, for a specific person or, in the case of standard shapes, of fitting only in limited manner.

An improved pressure distribution and shape adaptation, particularly for utility objects that relate to the support apparatus of human beings, is achieved by means of the use of usual gels as the cushioning medium.

DE 298 16 352 U1 concerns itself with the use of bags filled with gel, for shoe insert soles. By using plastic films that can be bonded and are highly tear-resistant, a corresponding pressure stress capacity and non-sensitivity to cold is guaranteed.

DE 200 11 334 U1 also concerns itself with the use of a gel cushion as a damping element in a shoe insert sole.

For safety work shoes, the use of gel cushions as a damping element is described in EP 0 774 219 A1.

WO 96 28 057 describes a shoe sole having a particular structure, which allows improved form fitting on the basis of the use of gels. The principle is based on a sandwich structure of the shoe sole, consisting of an outer layer, an inner layer, and an intermediate layer. The intermediate layer consists of two types of gel. The first type of gel, having viscoelastic properties, is a usual polyurethane gel, for example. The second type of gel, arranged underneath that, is a temperature-sensitive gel demonstrating lower critical solution temperature behavior. This gel is heated under the effect of the foot temperature, and gradually adapts to the foot shape of the user by swelling or shrinking as it is being worn.

All of the aforementioned gel-based utility objects avoid local pressure peaks by taking advantage of the viscoelastic properties of gels, which guarantee very good adaptation to the body and a very good pressure distribution. Their significant disadvantage consists in the exclusive use of the principle of soft contact. The problem of the constant stress on one and the same contact point remains unsolved. Also, in the case of the shoe sole according to WO 96 28 057, the adaptation to the body shape of the foot only takes place gradually.

### PRESENTATION OF THE INVENTION

It is the task of the invention to develop an intermediate layer for optimally enhancing the comfort of a user wearing intensively utilized utility objects on or in the body, for human beings or for animals.

According to the invention, this task is accomplished by means of the characterizing features indicated in claim 1. Advantageous embodiments are indicated in claims 2 to 13.

The invention relates to applications that are based on the effect of active surrounding and cushioning using known, temperature-sensitive gels. Temperature-sensitive gels are classified in the class of so-called smart gels. The particular unique feature of smart gels consists in the fact that they reversibly change their properties, particularly their volume, i.e. their degree of swelling, even at slight changes of a certain environmental variable, because of their phase transition behavior. The swelling equilibrium adjusted by the smart gel, in each instance, can be assigned to a specific quantity of the environmental variable. Temperature-sensitive gels having a lower critical solution temperature (LCST) behavior are relevant for the invention. Gels having LCST characteristics swell at temperatures below the phase transition temperature, and liquefy when the phase transition temperature is exceeded.

According to the invention, this behavior is utilized for an active intermediate layer having at least one component consisting of temperature-sensitive gels, for enhancing the comfort of a user wearing utility objects on or in the body. If the user wears the utility object on or in the body, the skin temperature, i.e. body temperature is transferred to the intermediate layer having the temperature-sensitive gel, because of the direct contact at the contact points. If the phase transition temperature is slightly below the skin temperature, i.e. body temperature, the phase transition temperature of the gel is exceeded, and it liquefies locally at this point. This results in the formation of new contact points and to a relief of stress at the old point. In an extreme case, the latter can be completely free of contact. This process repeats itself at the new contact points, while the old contact point cools off again, as a result of the reduced heat transfer, and swells again once the temperature goes below the phase transition temperature. Of course, temperature-sensitive gels also possess the advantageous viscoelastic properties of conventional gels, and therefore additionally guarantee very good soft contact.

The permanent relocation of the local contact points is a low-dynamic process, so that the user does not notice this effect. Depending on the use, the optimal relocation time can lie between 30 s and 2 h. Longer times should be avoided, since then the formation of painful pressure points must be expected, if the capillary occlusion pressure is exceeded. However, in the case of two-ply intermediate layers, the desired soft-dynamic relocation effect only occurs if the layer with the temperature-sensitive gel is arranged on the body side and if the body heat can control the gel behavior without hindrance, or practically without hindrance.

The value of the phase transition temperature of the temperature-sensitive gel being used is dependent on the body region in which the intermediate layer is supposed to be used, since the body possesses typical isotherms for the different body parts.

The intermediate layer is preferably an integral part of a utility object that is in constant use by the user, such as a prosthesis, a hearing aid, eyeglasses, a wig, dentures, or an orthopedic aid that must be permanently worn, or a utility object that is only worn temporarily or for a certain period of time, such as an orthopedic splint, an orthotic, a plaster cast, a wound dressing, a bandage or a support belt, a compress, compression stockings, compression pantyhose, orthopedic inserts, a support girdle.

Another area of application of the intermediate layer is the area of work safety, health protection, and accident prevention, for example in the case of safety masks, safety helmets, safety glasses, safety gloves, ankle, knee, wrist, and elbow guards, and safety shoes.

Another area of application of the intermediate layer is straps, for example tie straps, pull straps, carry straps, of course also for animals, bands, handles, carriers or supports of an article of clothing, or of another utility object to be worn. Examples are the shoulder straps or the frame of a backpack, the handle of a suitcase, or the ends of a bicycle handlebar, the shoulder strap or the padding of a bra, a wristwatch strap.

Another area of application is the use of the intermediate layer in invasive medicine, for example for catheters, ports, breathing tubes, implants.

Still another area of application is the use of the intermediate layer for saddles of animals to be ridden, whereby a temperature-sensitive layer can be arranged both on the side of the animal and on the side of the rider. Likewise, an intermediate layer on saddles of two-wheeled vehicles is useful.

### BRIEF DESCRIPTION OF THE DRAWINGS

The invention will be explained in greater detail, using several exemplary embodiments.

The related drawings show:

FIG. 1: a utility object in contact with the body, having an intermediate layer according to the invention, in different work phases,

FIG. 2: a fundamental structure of a utility object having an intermediate layer,

FIG. 3: a temple of a pair of eyeglasses,

FIG. 4: part of a hearing aid that is fitted inside the ear, and

FIG. 5: the fundamental structure of a safety helmet.

### BEST WAYS OF IMPLEMENTING THE INVENTION

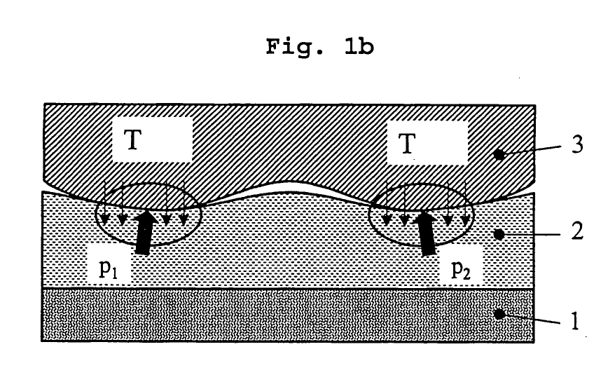

A utility object 1, not shown in greater detail in FIG. 1a, for example a hearing aid fitted inside the ear, is coated with an intermediate layer 2, which contains a temperature-sensitive gel, e.g. poly-(N-isopropyl acrylamide). It is practical if the combination of the inside-the-ear hearing aid 1 and the intermediate layer 2 has a standard shape, which is roughly adapted to the anatomy of the external ear and the auditory canal of its user. When it is worn, it is in contact with the body part 3 of the user, in other words with the inside of the ear and the external ear, in the example shown. Because the fit of the combination to the body part 3 is not ideal, the contact point will possess local regions against which the actual contact points lie. At these contact points, pressures p₁ and p₂ occur, caused, for example, by the inherent weight of the inside-the-ear hearing aid, which put stress on the skin of the body part 3. If the body temperature T of the body part 3 is transferred to the intermediate layer 2 for a long enough period of time (FIG. 1b), the gel of the intermediate layer 2 is heated above its phase transition temperature of 34° C., for example, and liquefies locally at these points. As FIG. 1c shows, this relieves the stress on the old contact points (in an extreme case, by eliminating the contact), and new contact points are formed, at which the pressures p₃, p₄ and p₅ then occur. After some time, the old contact points cool down below their phase transition temperature again, and swell up. In contrast, the process described repeats itself at the new contact points. Poly-(N-isopropyl acrylamide) is a temperature-sensitive hydrogel having a phase transition temperature of 33° C. to 34° C., and is therefore suitable for very many intermediate layers according to the invention, without additional treatment. In known manner, however, the value of the phase transition temperature can be adjusted, in targeted manner, to values between 5° C. and 50° C., and therefore to practically any case of use covered by the invention, by means of copolymerization or a variation of the gel composition.

The intermediate layer 2 offers a practical and attractive increase in the wearing comfort and use comfort for the areas of use mentioned in the dependent claims. However, they are not treated exhaustively, by any means, since the use of the intermediate layer is also recommended for many other applications.

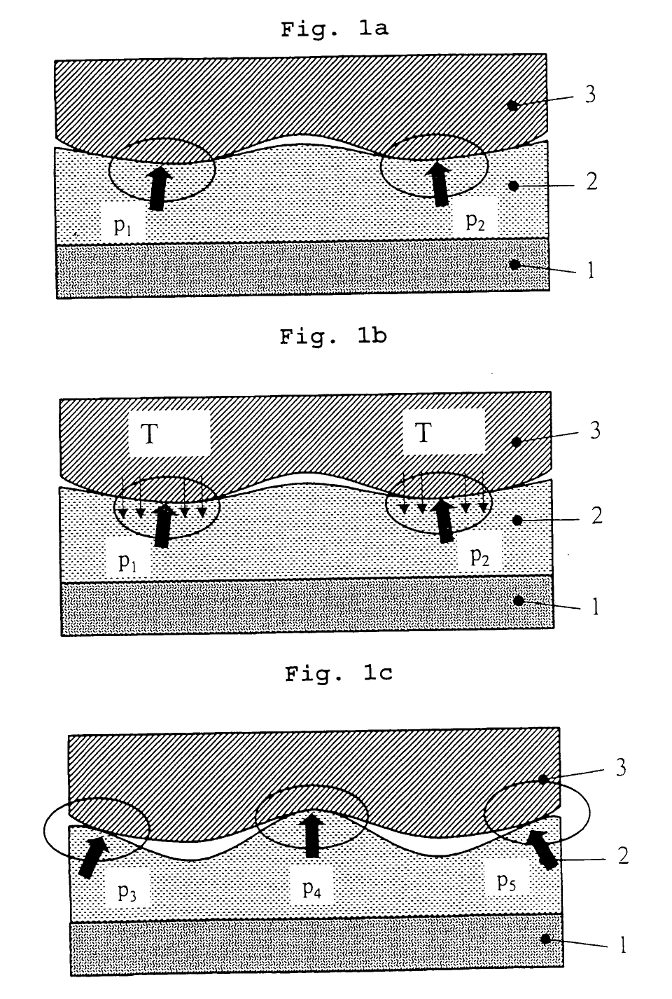

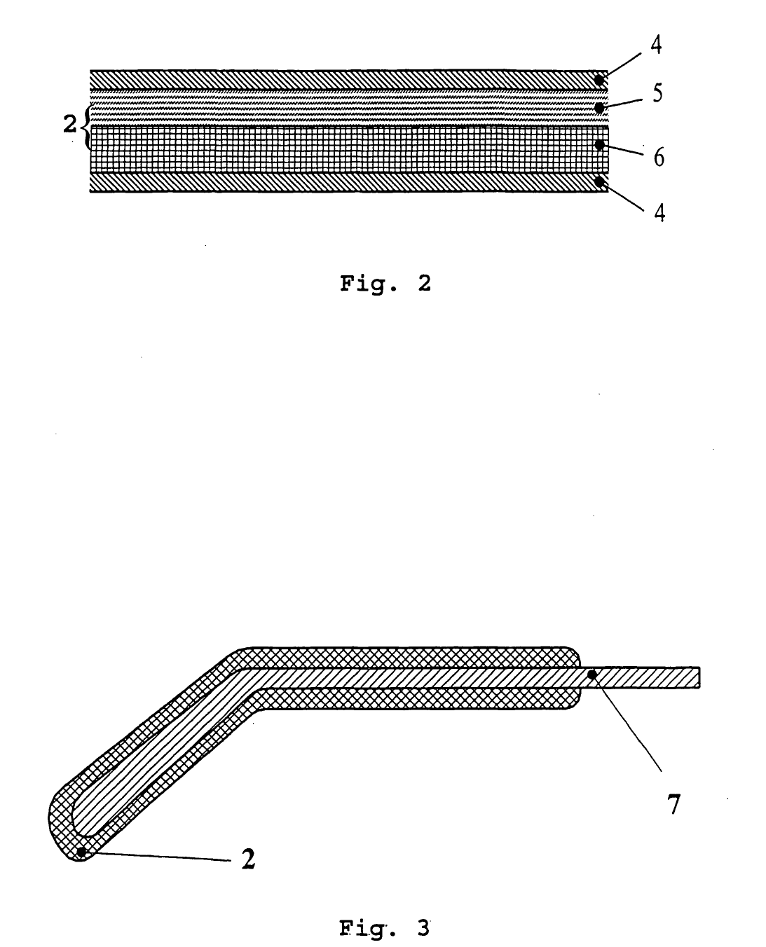

The intermediate layer 2 has the fundamental structure shown in FIG. 2. It is surrounded by a sheath 4, which is produced from a thin material that is compatible with the body and is sufficient to withstand the mechanical stresses. It can contain not only elastics but also textile components, since materials that are sufficiently thin do not impair the required heat transfer for any lasting period of time. On the side that faces the body of the user, the active layer 5, which consists of a temperature-sensitive gel, e.g. polyvinyl methyl ether, follows the intermediate layer 2. Polyvinyl methyl ether has a phase transition temperature of 37° C. The active layer 5 can be chambered, particularly for very large intermediate layer areas, for reasons of the effects of the heat capacity, so that local heating of one or a few chambers is possible. Chambering is not necessary for areas that are smaller than 1 cm³, since heat capacity effects are of subordinate importance here. The active layer 5 should be configured in such a manner that water that is released in the case of local liquefaction can be distributed within the space provided.

Particularly in the case of very large-area applications, the soft contact effect that is based on the viscoelastic gel properties can be reinforced by an additional layer 6, which consists of conventional gels, such as polyurethane gel, for example. This non-temperature-sensitive layer 6 of the active intermediate layer 2 is located on the side of the intermediate layer 2 that faces away from the body.

The active intermediate layer 2 according to FIG. 2 can also be configured without the additional layer 6, for example, for space reasons.

In the example according to FIG. 3, the mechanical stability of the active intermediate layer 2 is guaranteed by a temple part 7 of a pair of eyeglasses. The temple part 7 is sheathed by the active intermediate layer 2 in the region of the pressure points at which contact with the user's head occurs. In the case of eyeglasses, other pressure points are found, for example, in the nose region. Such frame parts of eyeglasses, for the sides or bridge of the nose, can also be equipped with an active intermediate layer 2, analogously.

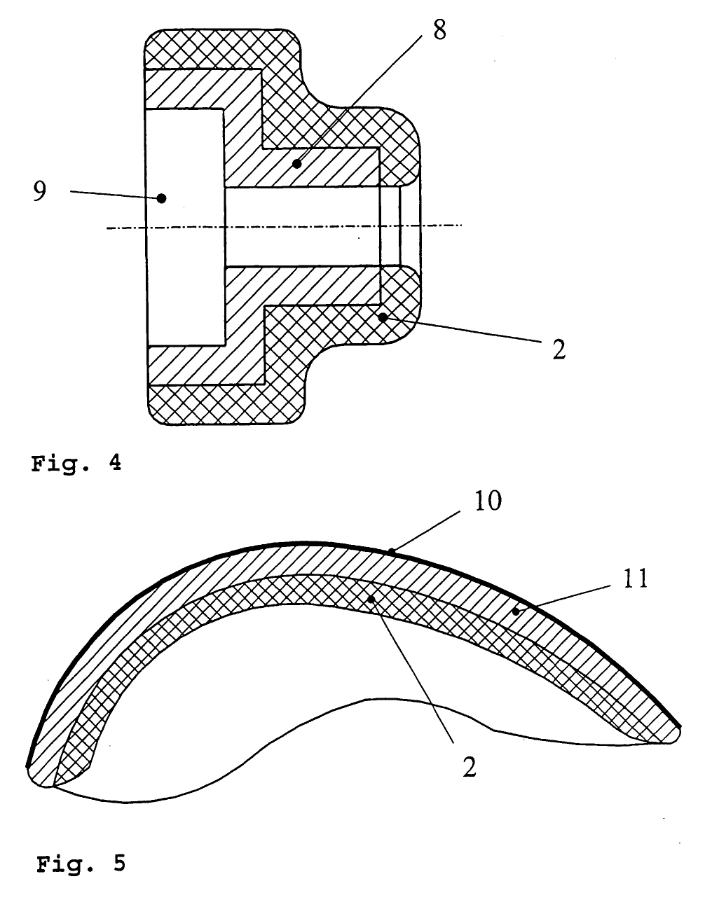

FIG. 4 schematically shows a cross-section through part of an inside-the-ear hearing aid. Again, the housing 8 is sheathed with an active intermediate layer 2 and provides mechanical stability. The cavity 9 serves to hold the loudspeaker unit. Of course the active intermediate layer 2 can also be anatomically shaped.

FIG. 5 schematically shows a cross-section through a safety helmet, for example a bicycle helmet. The safety helmet is composed of a mechanically stable outer layer 10, an inactive cushioning layer 11, and an active intermediate layer 2 on the side of the head. An improved fit, an improved hold, and better pressure distribution on the user's head result from active adaptation to the shape.

1. Intermediate layer for enhancing the comfort of a user wearing utility objects on or in the body, having at least one component consisting of a temperature-sensitive gel demonstrating LCST behavior (lower critical solution temperature behavior), wherein the intermediate layer (2) actively brings about a continuous change of the contact point(s) of the utility object with the body (3) of the user, by means of the influence of the body temperature, i.e. skin temperature (T) of the user.

2. Intermediate layer according to claim 1, characterized in that
the intermediate layer (2) is part of a utility object to be worn permanently or for an extended period of time.

3. Intermediate layer according to claim 2, characterized in that
the intermediate layer (2) is part of a prosthesis, a hearing aid, eyeglasses, a wig, or dentures.

4. Intermediate layer according to claim 1, characterized in that
the intermediate layer (2) is part of a surgical or orthopedic utility object.

5. Intermediate layer according to claim 4, characterized in that
the intermediate layer (2) is part of a splint, an orthotic, a plaster cast, a wound dressing, a bandage, a support belt, a support girdle, a compression stocking, a compress, or an orthopedic insert.

6. Intermediate layer according to claim 1, characterized in that
the intermediate layer (2) is part of a utility object from the sector of work safety, health protection, and accident prevention.

7. Intermediate layer according to claim 6, characterized in that
the intermediate layer (2) is part of a safety mask, a safety helmet, safety glasses, a safety glove, or an ankle, knee, wrist or elbow guard.

8. Intermediate layer according to claim 1, characterized in that
the intermediate layer (2) is part of an article of clothing or an article to be worn, which comes into contact with a body part of the user.

9. Intermediate layer according to claim 8, characterized in that
the intermediate layer (2) is part of a strap, a band, a wristwatch strap, a handle, a carrier, or a support.

10. Intermediate layer according to claim 1, characterized in that
the intermediate layer (2) is part of a utility object from the sector of invasive medicine.

11. Intermediate layer according to claim 10, characterized in that
the intermediate layer (2) is part of a catheter, a port, a breathing tube, or an implant.

12. Intermediate layer according to claim 1, characterized in that
the intermediate layer (2) is part of a saddle.

13. Intermediate layer according to claim 12, characterized in that
the intermediate layer (2) is part of a saddle for an animal to be ridden or of a saddle for a two-wheeled vehicle.
